(19) United States
(12) Patent Application Publication
(10) Pub. No.: US 2019/0038042 A1
(43) Pub. Date: Feb. 7, 2019

(54) Bed enabling simulation of an absence of gravity, sensorial simulation and/or isolation device incorporating said bed

(71) Applicant: OFF MATRIX HOLDINGS, Geneve (CH)

(72) Inventor: Denis BEDAT, Gujan-Mestras (FR)

(21) Appl. No.: 16/076,408

(22) PCT Filed: Feb. 10, 2017
(86) PCT No.: PCT/EP2017/052972

§ 371 Date: Aug. 8, 2018

(30) Foreign Application Priority Data
Feb. 11, 2016 (FR) 1651099

(51) Int. Cl. A47C 27/08; A61M 21/00

(52) U.S. Cl. CPC A47C 27/085; A61G 10/023; A61M 21/0094

(57) ABSTRACT

A bed making it possible to simulate an absence of gravity comprises a tank containing a fluid with a specific gravity of between 1.20 and 1.23, a plate mobile between a high position above the fluid and an immersed low position, and a membrane covering the plate and having a thickness less than or equal to 0.3 mm A sensory isolation and/or stimulation chamber incorporates such a bed.

### CROSS-REFERENCES TO RELATED APPLICATIONS

This application claims the benefit of the French patent application No. 1651099 filed on Feb. 11, 2016, the entire disclosures of which are incorporated herein by way of reference.

### BACKGROUND OF THE INVENTION

The present application relates to a bed that makes it possible to simulate an absence of gravity and a sensory isolation and/or stimulation device incorporating the bed.

A sensory isolation chamber is a tank containing salt water covered by a lid which forms, with the tank, an enclosure. In practice, an individual lies down in the water which is heated to the skin temperature of the individual (of the order of 37° C.) and the lid is closed.

This type of sensory isolation chamber is not fully satisfactory because the skin of the individual is in contact with the salt water which can prove problematic for some individuals, or in certain circumstances, and which necessitates cleaning to remove the salt deposited on the skin. According to another issue, it necessitates a water treatment system to maintain the quality of the water from one individual to another.

The document WO2012/098076 proposes a device for immersing an individual in water without contact with the water. This device comprises a tank containing fresh water, a plate which is mobile heightwise between a first position out of the water and an immersed position and a lid which covers the plate, and which is linked to the tank all along its periphery. Thus, the lid forms a barrier between the individual and the water.

According to this document, the lid has a thickness of between 0.15 and 0.5 mm It also comprises an excess length so as to envelop the individual when the plate is immersed, without that exerting a load on the link between the lid and the tank.

To raise and lower the plate, the device comprises an immersed scissor lifting system, interposed between the bottom of the tank and the plate.

This device is not fully satisfactory for the following reasons:

Upon immersion, given the distribution of the weight of the body, the individual does not remain approximately horizontal but is inclined in the water, the head being positioned out of the water while the lower limbs are arranged almost at the bottom of the tank. This posture is uncomfortable because the part of the body of the individual that is immersed is compressed. Because of this, the individual has difficulty in breathing and the blood pressure is out of balance, the pressure in the lower limbs being significantly greater than that of the head so that the heart has to make a greater effort to circulate the blood to the head. Consequently, the expansion of the neuromuscular system is not optimal.

Given the discomfort imparted by this device, a cushion is generally provided under the head of the individual. Because of this, the head of the individual does not feel the absence of gravity.

According to another drawback, the link between the lid and the tank being tight, the appearance of air bubbles under the lid is observed upon immersion which disrupts the simulation of absence of gravity.

According to another drawback, this device is uncomfortable when the person is not immersed.

The present invention aims to remedy the drawbacks of the prior art.

### SUMMARY OF THE INVENTION

To this end, the subject of the invention is a bed which makes it possible to simulate an absence of gravity and which comprises:
a tank containing a fluid with a specific gravity of between 1.20 and 1.23;
a plate that is mobile between a high position above the fluid and an immersed low position;
a membrane which covers the plate, and which has a thickness less than or equal to 0.3 mm

The combination of a membrane with a thickness less than 0.3 mm and of a specific gravity for the fluid of between 1.20 and 1.23 makes it possible to obtain an optimal simulation of weightlessness.

Preferably, the membrane is linked at the periphery to the tank and has excess lengths equal to at least 25% of the dimensions of the tank. Thus, when the plate is in low position, the excess lengths make it possible to avoid having the membrane subjected to tensions likely to disrupt the simulation of weightlessness.

Advantageously, the membrane has a modulus of elasticity less than or equal to 100 MPa. This feature gives the membrane a great flexibility together with its fine thickness which makes it possible to optimize the sensation of weightlessness.

According to another feature, the bed comprises a mattress interposed between the plate and the membrane and linked to the plate. The presence of the mattress improves the comfort of the bed when the plate is not immersed. The fact that it is linked to the plate makes it possible, when the plate is in immersed position, to avoid having the mattress disrupt the simulation of weightlessness.

According to another feature, the bed comprises a mechanism for displacing the plate being positioned above the plate.

### BRIEF DESCRIPTION OF THE DRAWINGS

Other features and advantages will emerge from the following description of the invention, the description being given by way of example only, in light of the attached drawings in which:

FIG. 1 is a longitudinal cross section of a bed which schematically illustrates all embodiments of the invention, without an individual,

FIG. 2 is a longitudinal cross section of the bed of FIG. 1, with an individual, in a high position,

FIG. 3 is a longitudinal cross section of the bed of FIG. 1, with an individual, in a low position,

FIGS. 4A and 4B are transverse cross sections of a bed which schematically illustrate the invention without the individual and with the individual, in the low position,

FIG. 5 is a cross section of a top part of the side of a bed which illustrates an embodiment of the invention,

FIG. 6 is a plan view of a corner of the bed which illustrates an embodiment of the invention,

FIG. 7 is a perspective view of a mobile plate added above a bed which illustrates an embodiment of the invention,

FIG. 8 is a perspective view of a structure of canopy type added above a bed which illustrates an embodiment of the invention,

FIGS. 9A and 9B are perspective views of a sensory isolation and/or stimulation device which illustrate an embodiment of the invention in the open state and in the closed state.

### DETAILED DESCRIPTION OF THE PREFERRED EMBODIMENTS

In the different figures, a bed making it possible to simulate the absence of gravity is represented as 10.

This bed 10 comprises a tank 12 with a bottom 14 and at least one lateral wall, the top part being free. According to one configuration, the tank is parallelepipedal and comprises two right and left lateral walls 16, two front and rear end walls 18. However, the invention is not limited to this geometry.

To give an order of magnitude, the tank has a height of the order of 0.7 m, a width of the order of 1 m and a length of the order of 2.4 m. These dimensions correspond to the outer dimensions of the tank.

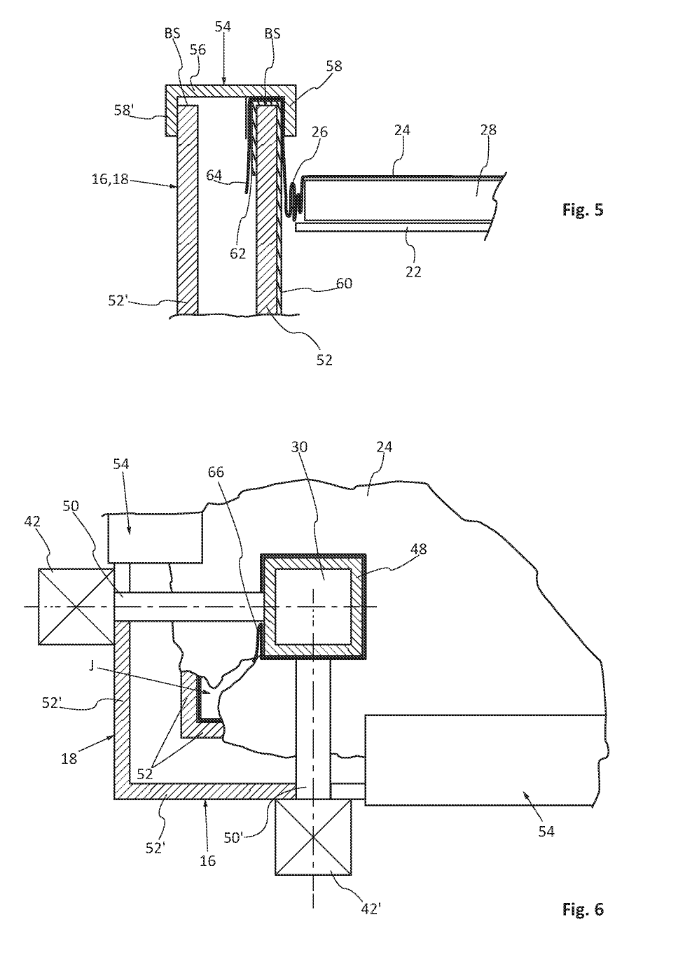

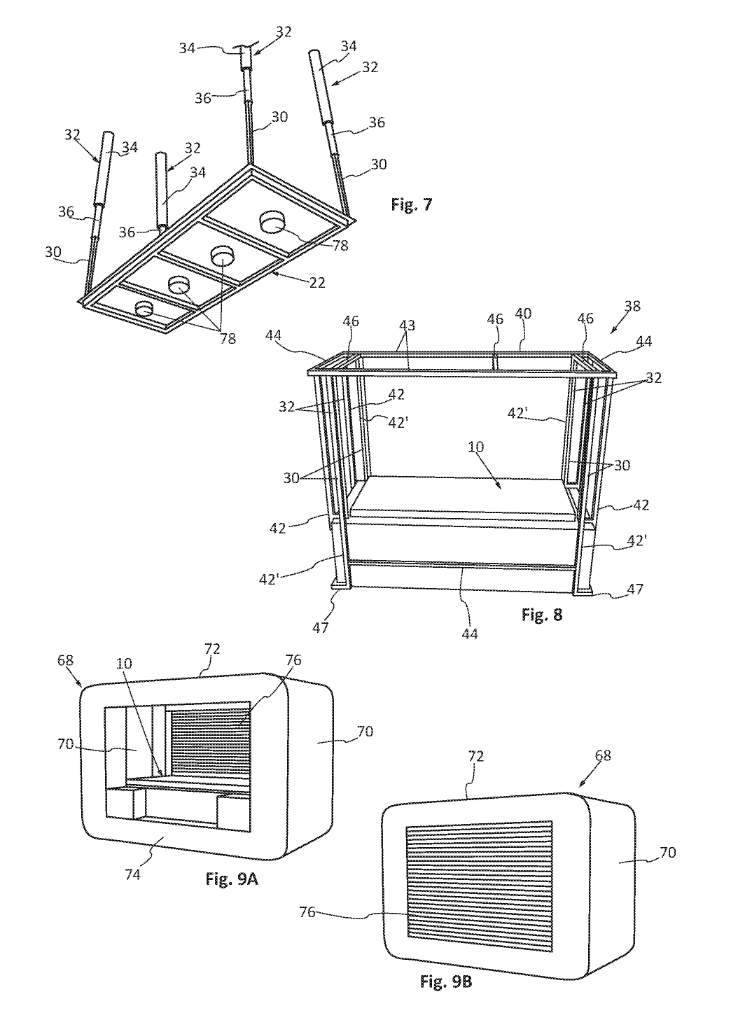

The lateral and end walls have a top edge BS visible in detail in FIG. 5.

The tank 12 contains a fluid 20 with a specific gravity of between 1.1 and 1.3. According to an embodiment of the invention, the density lies between 1.20 and 1.23.

According to one embodiment, the fluid contained in the tank is salt water whose salinity is adjusted so as to obtain a specific gravity of between 1.20 and 1.23. Optimally, the specific gravity of the fluid 20 is of the order of 1.215.

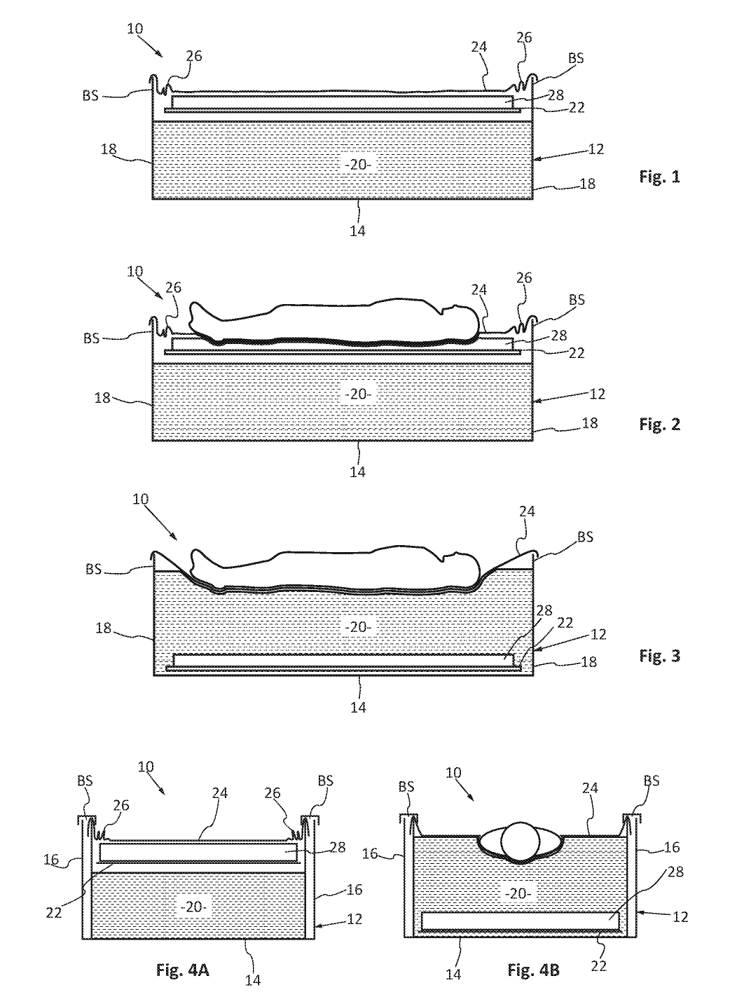

The bed comprises a horizontal plate 22, mobile vertically between a high position (visible in FIGS. 1, 2, 4A) in which the plate 22 is situated above the fluid 20 and a low position (visible in FIGS. 3 and 4B) in which the plate 22 is immersed.

This plate 22 is metal and made of a material resistant to water with high salinity. According to one embodiment, the plate is made of an aluminum alloy of marine quality.

The plate 22 has a geometry and dimensions matched to that of the tank 12 so that there remains a gap at the periphery between the plate and the tank of the order of 1 to 4 cm. In the case of a parallelepipedal tank, the plate has a width of the order of 0.9 m and a length of the order of 2.2 m.

The bed 10 also comprises a membrane 24 which covers the plate 22 and which is linked at the periphery to the tank 12, notably at the top edges BS.

The membrane 24 has to be as thin as possible to come into contact with an individual when the latter is lying on the bed and the plate is in low position.

Preferably, the membrane 24 is impervious to the fluid. It is made of a material allowing contact with the skin.

According to an important feature of the invention, the membrane 24 has a thickness less than or equal to 0.3 mm

The combination of a membrane with a thickness less than 0.3 mm and a specific gravity for the fluid of between 1.20 and 1.23 makes it possible to obtain an optimal simulation of weightlessness. The presence of the membrane makes it possible to avoid having the individual be in contact with the water with high salinity. According to the invention, neither the specific gravity of the fluid alone, nor the thickness of the membrane alone make it possible to obtain an optimal buoyancy. Thus, the specific gravity of the fluid is matched to this membrane thickness. Thus, the range of specific gravity is slightly less than that of the salt water of the sensory isolation chambers of the prior art to take account of the membrane 24 and not obtain an excessive buoyancy. Furthermore, the provision of salt water under the membrane 24 makes it possible to reinforce the buoyancy compared to the device described in the document WO2012/098076 which provides for only fresh water.

According to another feature, the membrane 24, has dimensions such that there are excess lengths 26 distributed at the periphery of the membrane 24 when the plate 22 is in the high position. These excess lengths make it possible to avoid having the membrane subjected to tension when the plate is in low position. Thus, the membrane according to the invention has almost no effect on the buoyancy

and serves only as a barrier between the fluid and an individual lying down on the bed.

Advantageously, the membrane 24 has dimensions such that the excess lengths represent, for a dimension (length or width) at least 25% of the corresponding internal dimension of the tank.

As an indication, for a tank with dimensions as mentioned previously, the membrane is a rectangle measuring 2 m×3 m.

According to another feature of the invention, the membrane 24 has to be as flexible as possible so as not to be felt by the individual and thus optimize the sensation of an absence of gravity. To this end, the membrane 24 preferably has a modulus of elasticity less than or equal to 100 MPa. Optionally, the membrane has a modulus of elasticity less than or equal to 20 MPA.

According to an embodiment, the membrane 24 is made of latex, has a thickness of the order of 0.2 mm and the following properties:

tensile strength: 25 MPa;

modulus of elasticity: 3 MPa;

elongation at break: 850%;

tearing strength: 70 N/mm

Obviously, the invention is not limited to these properties for the membrane 24.

According to another feature, a mattress 28 is interposed between the plate 22 and the membrane 24.

Preferably, the mattress 28 is an air mattress. Advantageously, it comprises a plurality of independent cells arranged in one and the same plane along several lines and several rows. The cells all have almost the same form, cubic or parallelepipedal.

The mattress 28 is made of a material suited to the saline medium. As an example, the mattress is made of polychloroprene-based elastomer.

According to an embodiment, the mattress is of bariatric type.

The presence of the mattress makes it possible to enhance the comfort of the bed when the plate 22 is in the high position and is not immersed. Thus, the individual lying down on the bed is not lying down on a rigid element but on a soft element.

Advantageously, the mattress 28 is linked to the plate 22 so as not to float when the plate 22 is in the low position and immersed. According to an embodiment, straps are used to link the mattress 28 to the plate 22. This configuration makes it possible to avoid having the mattress disturbed by the effect of the absence of gravity.

According to another feature, the bed comprises a plate displacement mechanism 22, positioned above the plate 22. According to this configuration, the displacement mechanism is not immersed in the water with strong salinity which makes it possible to simplify the design thereof.

Advantageously, the plate 22 comprises at least one upright 30 which extends from the top face of the plate, at right angles to the face, and at least one actuator 32 configured to displace the upright 30 vertically in two opposite directions.

According to one configuration, the plate 22 comprises four uprights located at each corner of the plate. Of course, the invention is not limited to this configuration.

The arrangement of the uprights 30 is linked to the geometry of the plate.

Preferably, the plate 22 comprises at least three uprights which confers a better stability.

The displacement mechanism comprises as many actuators 32 as uprights.

Preferably, each actuator 32 is a cylinder which comprises a body 34, relative to which slides a rod 36 arranged in the extension of the upright 30.

According to this configuration, the bed 10 comprises a supporting structure 38 of a canopy type which caps the bed 10, each actuator 32 being interposed between the supporting structure 38 and an upright 30.

According to an embodiment, the uprights 30 are produced in the same material as the plate 22 and are linked to the plate 22 by any appropriate means, such as, for example, by welding or riveting.

According to a preferred configuration, each actuator 32 is an electrical cylinder.

The bed 10 comprises a control for driving the actuators and triggering the lowering or the raising of the plate 22. Preferably, this control comprises a safety device for controlling the raising of the plate 22 and imposing a slow raising speed less than 0.5 m/min. This solution makes it possible to restore the effect of gravity very gradually.

According to an embodiment illustrated in FIG. 8, the supporting structure 38 is a mechanically welded structure which comprises an upper frame 40 positioned above the tank 12 and supported by feet 42.

The frame 40 comprises, in addition to the long sides 43 and the short sides 44, cross members 46 which link the long sides 43 and which are parallel to the short sides 44. These cross members 46 are notably positioned vertically below the uprights 30 so as to support the actuators 32. Other cross members can be provided, to, for example, support a screen.

As illustrated in FIG. 6, for each corner of the bed, the supporting structure 38 comprises two feet, a first foot 42 pressed against the outer face of an end wall 18 and a second foot 42′ pressed against the outer face of a lateral wall 16. According to an embodiment, the two feet 42′ pressed against the same lateral wall 16 are linked by a stringer 44 parallel to the ground and offset heightwise.

The two feet 42′ pressed against two different lateral walls and the two feet 42 pressed against the same end wall are linked by U-shaped reinforcement 47, positioned at ground level.

Obviously, the invention is not limited to this architecture for the supporting structure 38.

According to another feature of the invention, the bed 10 comprises a guide 48 for each upright 30, the guide 48 being fixed relative to the tank 12.

According to a configuration, each upright 30 is a profile member with a square or rectangular section. In this case, each guide 48 is a hollow tube with a passage section identical to the sliding clearance of the outer section of the upright 30.

The guides 48 are arranged approximately in the same horizontal plane, just above the top edges of the walls 16 and 18 of the tank 12. Each guide 48 is positioned at a corner of the tank 12. It is linked to the feet 42, 42′ by two rods 50, 50′, a first rod 50 linking the guide 38 to a first foot 42 and a second rod 50′ linking the guide 38 to a second foot 42′.

The guides 38 limit the movements of the plate 22 in a horizontal plane when an individual is settled on the bed 10.

For each guide/upright pair, the upright and/or the guide is/are produced in a material or comprises/comprise a coating of a material which promotes the sliding of the upright 30 in the guide 38.

According to a configuration illustrated in FIGS. 5 and 6, each wall 16, 18 of the tank comprises two plates 52, 52′ that are parallel and spaced apart on the order of a few centimeters, for example from 3 to 6 cm. Thus, each wall 16, 18 comprises an inner plate 52 oriented toward the interior of the tank and an outer plate 52′ oriented toward the exterior of the tank.

Preferably, each wall 16, 18 of the tank 12 comprises a U-shaped angle iron 54 which caps the top edge BS of the wall 16, 18. In the case of walls 16 and 18 with two parallel plates 52, 52′, each angle iron 54 has a base 56 whose width is matched to the thickness of the wall 16, 18 so that a first leg 58 of the angle iron 54 comes into contact with the inner face of the inner plate 52 and a second leg 58′ of the angle iron 54 comes into contact with the outer face of the outer plate 52′.

Preferably, the tank 12 comprises a liner 60 which covers the bottom 14 of the tank and rises up along the lateral 16 and end 18 walls. Preferably, this liner 60 covers the top edge BS of the walls 16, 18 and is held by the angle irons 54 which cap the top edge BS of the walls 16, 18. In the case of walls with two parallel plates 52, 52′, the excess 62 of the liner 60 is arranged between the two plates 52, 52′ and the liner 60 is pinched between the angle irons 54 and the top edge BS of the inner plate 52 of the walls 16, 18.

With an angle iron 54, the membrane 24 is held at the periphery of the tank 12 by the angle irons by being pinched between the angle irons and the top edge of the walls 16, 18. In the case of walls 16, 18 with two parallel plates 52, 52′, the excess 64 of the membrane 24 is arranged between the two plates 52, 52′ and the membrane 24 is pinched between the angle irons 54 and the top edge BS of the inner plate 52 of the walls 16, 18.

As illustrated in FIG. 6, the angle irons 54 are away from the corners of the tank. According to another point, the guides 38 are separated from the walls 16, 18, so that there remains a gap J between the guides 38 and the walls 16 and 18. Since the membrane 24 is not held by the angle irons 54 at the gap J provided between the guides 38 and the walls 16 and 18, the volume delimited by the tank 12 and the membrane 24 communicates with the outside. This configuration makes it possible to limit the appearance of air bubbles under the membrane 24 during the lowering of the plate 22 which are likely to detract from the simulation of absence of gravity.

At the corners of the tank, the membrane 24 is linked all around the guides 38 by virtue of a clamping collar 66.

The bed 10 comprises water heating means and regulation means for controlling the heating means in order to heat up and maintain the temperature of the water at a temperature close to that of the human body. A temperature sensor can be provided to equip the individual lying down on the bed and inform the regulation means.

According to an application, the bed 10 is incorporated in a sensory isolation chamber 68.

This sensory isolation chamber 68 comprises an enclosure in which the bed 10 is positioned, the enclosure comprising at least one opening which can be blocked by a mobile element so as to open the enclosure to allow an individual to enter into the enclosure to lie down on the bed.

According to an embodiment, the sensory isolation chamber 68 comprises the supporting structure 38 and opaque walls 70 at the end walls 18 and an opaque wall 72 which forms the ceiling. Steps 74 are provided on either side of the bed, attached to the lateral walls to improve the access to the bed. Windable curtains 76 are provided approximately plumb with the lateral walls, at the ceiling 72, and are configured so as to close the lateral faces of the chamber 68 by being wound down. Thus, when at least one of the two curtains 76 is wound up, it allows access to the bed 10. When the two curtains 76 are wound down, the sensory isolation chamber 68 is closed. As a variant, the sensory isolation chamber 68 comprises a single windable curtain 76 on just one lateral face, the other lateral face being blanked off by a fixed opaque wall.

The sensory isolation chamber 68 comprises at least one sensory stimulation means so as to obtain a sensory isolation and stimulation chamber.

As an example and in a nonlimiting manner, the chamber comprises:

at least one image emitter (for example a screen) positioned on the ceiling, and/or

at least one sound emitter positioned in the fluid and/or outside of the fluid, and/or

at least one vibration emitter 78 attached to the plate 22.

As is apparent from the foregoing specification, the invention is susceptible of being embodied with various alterations and modifications which may differ particularly from those that have been described in the preceding specification and description. It should be understood that I wish to embody within the scope of the patent warranted hereon all such modifications as reasonably and properly come within the scope of my contribution to the art.

1-14. (canceled)

15. A bed making it possible to simulate an absence of gravity and comprising:
a tank containing a fluid,
a plate mobile between a high position above the fluid and an immersed low position,
a membrane covering the plate and having a thickness less than or equal to 0.3 mm,
a specific gravity of the fluid being between 1.20 and 1.23.

16. The bed as claimed in claim 15, wherein the membrane is linked at a periphery to the tank and has an excess length and width equal to at least 25% of a length and width of the tank.

17. The bed as claimed in claim 15, wherein the membrane has a modulus of elasticity less than or equal to 100 MPa.

18. The bed as claimed in claim 15, further comprising a mattress interposed between the plate and the membrane, and linked to the plate.

19. The bed as claimed in claim 15, further comprising a mechanism for displacing the plate being positioned above the plate.

20. The bed as claimed in claim 19, wherein the plate comprises at least one upright and wherein the plate displacement mechanism comprises at least one actuator configured to displace the at least one upright vertically in two opposite directions.

21. The bed as claimed in the claim 20, wherein the bed comprises a supporting structure, each actuator being positioned in an extension of an upright and being connected at one end to said upright and at another end to said supporting structure.

22. The bed as claimed in claim 20, wherein the bed comprises, for each upright, a guide fixed relative to the tank, arranged just above a top edge of walls of the tank, each guide comprising a hollow tube with a passage section identical to a sliding clearance of an outer section of the upright.

23. The bed as claimed in claim 15, wherein the tank comprises walls, each with an inner plate oriented toward an interior of the tank and an outer plate oriented toward an exterior of the tank, said plates being parallel and spaced apart.

24. The bed as claimed in claim 23, wherein the bed comprises, for each wall, a U-shaped angle iron, a first leg of said angle iron coming into contact against an inner face of the inner plate and a second leg of said angle iron coming into contact with an outer face of the outer plate.

25. The bed as claimed in the claim 24, wherein the membrane is pinched between the angle irons and a top edge of the walls.

26. The bed as claimed in claim 22,
wherein the tank comprises walls, each with an inner plate oriented toward an interior of the tank and an outer plate oriented toward an exterior of the tank, said plates being parallel and spaced apart, and
wherein the bed comprises, for each wall, a U-shaped angle iron, a first leg of said angle iron coming into contact against an inner face of the inner plate and a second leg of said angle iron coming into contact with an outer face of the outer plate, and
wherein the membrane is pinched between the angle irons and the top edge of the walls, wherein a gap is retained between each guide and the walls of the tank and
wherein the angle irons do not hold the membrane at said gap so that a volume delimited by the tank and the membrane communicates with the outside.

27. A sensory isolation and/or stimulation chamber comprising a bed as claimed in claim 15.

28. The sensory isolation and/or stimulation chamber as claimed in claim 27, further comprising at least one sensory stimulation means in the form of a vibration emitter attached to the plate.
